(19) United States
(12) Patent Application Publication
(10) Pub. No.: US 2013/0046335 A1
(43) Pub. Date: Feb. 21, 2013

(54) Dural repair instruments and methods of using the same

(71) Applicants: Lawrence S. Deutsch, Moorestown, NJ (US); Mark A. Testaiuti, Newtown Square, PA (US); Kamaldeep S. Momi, Medford, NJ (US)

(72) Inventors: Lawrence S. Deutsch, Moorestown, NJ (US); Mark A. Testaiuti, Newtown Square, PA (US); Kamaldeep S. Momi, Medford, NJ (US)

(21) Appl. No.: 13/199,024

(22) Filed: Aug. 17, 2011

(51) Int. Cl. A61B 17/28

(52) U.S. Cl. 606/205

(57) ABSTRACT

A needle holder instrument and needle for use in dural repair is disclosed along with a method for using the same. The needle holder instrument includes a handle portion that is adapted to be held by a surgeon for manipulating the instrument, a needle holder portion adapted to temporarily hold a needle and an elongated central portion extending between and connecting the handle portion to the needle holder portion. The needle holder portion includes a needle holder comprised of an aperture passing entirely through the needle holder portion adjacent the distal end thereof. The aperture is tapered whereby the diameter thereof is wider at the opening adjacent the surface of the needle holder portion and is narrower as the depth of the aperture increases. The needle has a pointed tip at one end thereof and a base at the other end thereof. The base has a shape that is complementary to the shape of the aperture whereby the base can be inserted into the aperture and temporarily held therein. Also provided are forceps including opposed working jaws having non-smooth surfaces that are adapted to grasp and hold the needle.

### BACKGROUND OF THE INVENTION

The present invention is directed toward dural repair instruments and methods and more particularly, toward such instruments and methods that are particularly adapted to assist a spine surgeon in suturing the dura that has either been torn or otherwise damaged or which has been intentionally incised during spinal surgery.

The dura mater (commonly referred to as the “dura”) is the outermost of the three layers of the meninges surrounding the spinal cord. The primary function of the dura is to protect, surround, and support the spinal cord. It forms the dural sac which extends from the foramen magnum all the way down to the coccyx. Inside this formed sac are the arachnoid mater, subarachnoid space, pia mater, and the spinal cord/nerves/roots.

The dura is also responsible for keeping in the cerebrospinal fluid. With the loss of spinal fluid, the pressure around the brain and spinal cord drops. The loss of spinal fluid can cause severe headaches, particularly when sitting up, and can result in serious infection. Infections after surgery or trauma can lead to meningitis and serious complications, such as swelling of the brain.

Leakage through the dura can be caused by trauma, spinal surgery, the placement of tubes for epidural anesthesia or pain medications and spinal taps (lumbar puncture). In many cases, the dura may eventually heal by itself with no lasting complications. In others, the dura must be repaired. This frequently requires that the tear or incision be sutured back together. This, however, can be a difficult procedure, even for skilled spine surgeons.

Because the surgical field is relatively small, it is frequently difficult to suture the dura using conventional suture needles and other instruments. This becomes particularly difficult with larger or obese patients where the distance to the dura is greater. The repair procedure requires that the surgeon have a clear view of the field and this becomes difficult with conventional instruments which interfere with the surgeon's line of sight. While bayonet shaped instruments have been proposed that may have some benefit, they do not totally satisfy the needs of the spine surgeon. One such bayonet shaped instrument is described, for example, in U.S. Pat. Nos. 6,962,582 and 7,163,532 that issued to Zinkel in 2005 and 2007, respectively.

Over the last decade or so spine surgery, like many other surgical fields, has moved towards more and more minimally invasive procedures. Spine surgeons today find they can do procedures that they traditionally did open through small tubes sometimes 14 mm in diameter and sometimes up to 120 mm in depth. Traditional instruments for dural repair are not designed for minimally invasive approach. In recent years with a dural tear surgeons would have to either struggle with improvised techniques or open the incision wider to use traditional instruments.

Inserting the suture needle into and pulling it through the dura is particularly difficult and conventional instruments provide little assistance. While needle drivers or inserters are known (see, for example, U.S. Pat. No. 1,037,864 to Carlson et al. that issued in 1912 and U.S. Pat. No. 4,161,951 that issued to Scanlan, Jr. in 1979) none is particularly useful in dural repair procedures and especially with minimally invasive procedures.

Thus, there is a need for a needle driver or inserter and other instruments that are particularly suited to assist a spine surgeon in suturing or otherwise repairing a dural tear or incision especially with minimally invasive procedures.

### SUMMARY OF THE INVENTION

The present invention is designed to overcome the deficiencies of the prior art discussed above. It is an object of the present invention to provide a needle driver or inserter and method that are particularly useful in suturing the dura through a minimally invasive approach.

It is another object of the present invention to provide a needle and suture that is particularly adapted to the needle driver of the invention.

It is yet another object of the present invention to provide forceps that are particularly adapted to grasp the needle of the invention.

It is an even further object of the present invention to provide a kit for dural repair that includes a needle driver, a needle and suture and forceps.

In accordance with illustrative embodiments demonstrating features and advantages of the present invention, there is provided a needle holder instrument and needle for use in dural repair. The needle holder instrument includes a handle portion that is adapted to be held by a surgeon for manipulating the instrument, a needle holder portion adapted to temporarily hold a needle and an elongated central portion extending between and connecting the handle portion to the needle holder portion. The needle holder portion includes a needle holder comprised of an aperture passing entirely through the needle holder portion adjacent the distal end thereof. The aperture is tapered whereby the diameter thereof is wider at the opening adjacent the surface of the needle holder portion and is narrower as the depth of the aperture increases. The needle has a pointed tip at one end thereof and a base at the other end thereof. The base has a shape that is complementary to the shape of the aperture whereby the base can be inserted into the aperture and temporarily held therein. Also provided are forceps including opposed working jaws having non-smooth surfaces that are adapted to grasp and hold the needle.

Other objects, features, and advantages of the invention will be readily apparent from the following detailed description of a preferred embodiment thereof taken in conjunction with the drawings.

### BRIEF DESCRIPTION OF THE DRAWINGS

For the purpose of illustrating the invention, there is shown in the accompanying drawings forms that are presently preferred; it being understood that the invention is not intended to be limited to the precise arrangements and instrumentalities shown.

FIG. 1 is a front elevational view of forceps useful with the present invention and shown in their closed position;

FIG. 2 is a front elevational view of forceps similar to FIG. 1 but shown in their open position;

FIG. 3 is an enlarged perspective view of the jaws of the forceps of FIGS. 1 and 2;

FIG. 4 is a perspective view of the needle holder or driver of the invention and showing a needle and suture positioned therein;

FIG. 5 is an enlarged perspective view of the end of the needle holder of FIG. 4 illustrating the details thereof;

FIG. 6 is an exploded view of FIG. 5 showing how the needle and needle holder interconnect;

FIG. 7 is a cross-sectional view of the needle base taken through the line 7-7 of FIG. 6;

FIG. 8 is a top plan view of the end of the needle holder through the line 8-8 of FIG. 6;

FIG. 9 is a cross-sectional view of the end of the needle holder taken through the line 9-9 of FIG. 6;

FIG. 10 illustrates the manner in which the instruments of the present invention are used for suturing and the beginning of the suturing process;

FIG. 11 illustrates the next step in the use of the instruments of the present invention, and

FIG. 12 illustrates a further step in the use of the instruments of the present invention.

### DETAILED DESCRIPTION OF THE PREFERRED EMBODIMENT

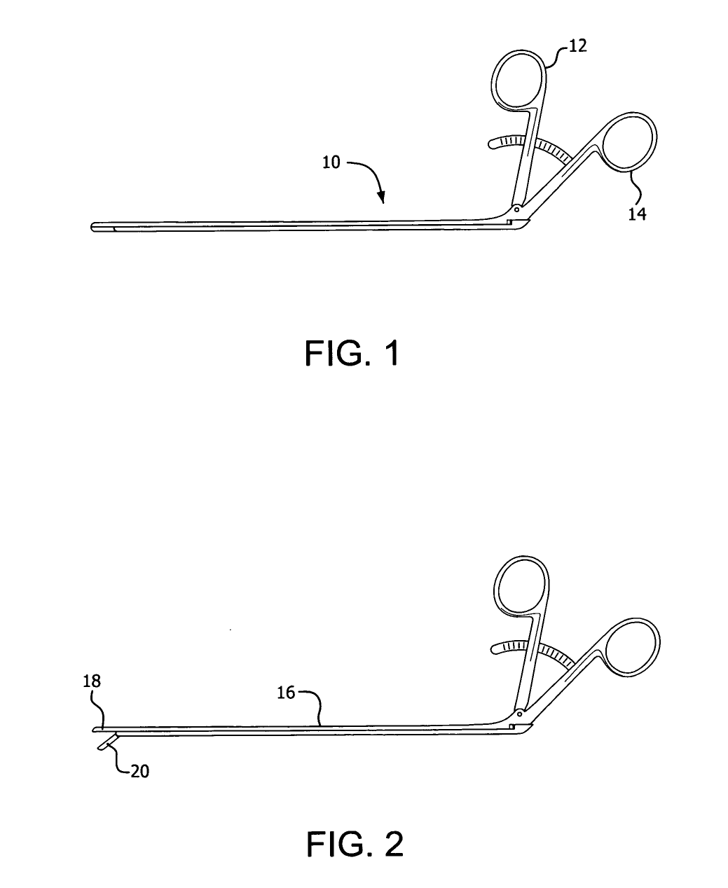

Referring now to the drawings in detail wherein like reference numerals have been used throughout the various figures to designate like elements, there is shown in FIG. 1 forceps constructed in accordance with the principles of the present invention and designated generally as 10. The forceps 10 are, in most ways, substantially identical to conventional forceps and similar instruments readily available in the market. The forceps 10 include handle portions 12 and 14 and an elongated shaft section 16 with opposed working jaws 18 and 20 at the distal end thereof. In a manner well known in the art, one or both of the jaws 18 and 20 can move toward and away from each other when the handles 12 and 14 are activated.

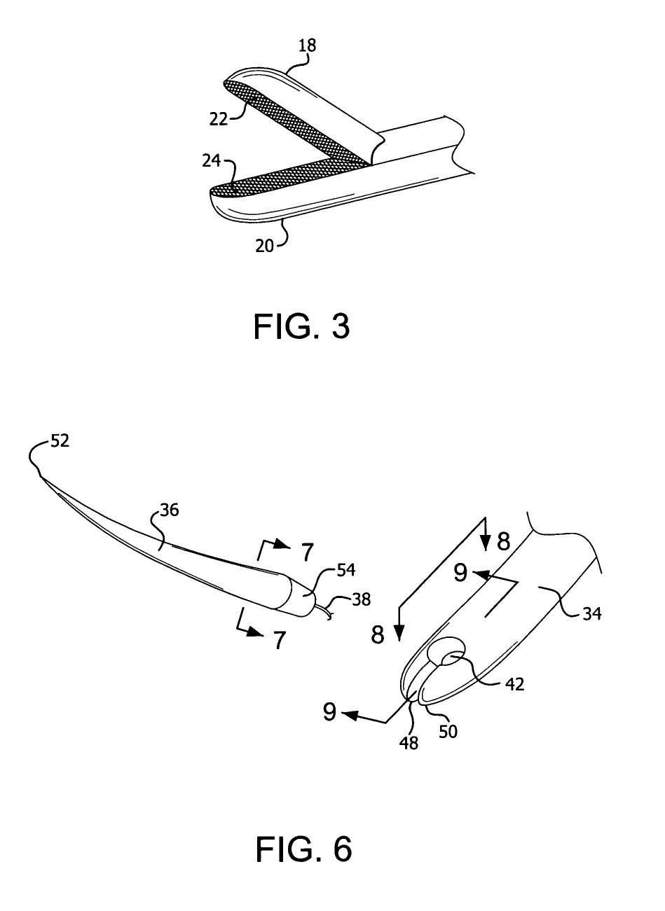

The primary difference between the forceps 10 of the present invention and conventional forceps is that the inside working surfaces of the jaws 18 and 20 have non-smooth surfaces such as shown at 22 and 24 in FIG. 3. The non-smooth surfaces are preferably formed by knurling or by providing a plurality of ridges or grooves in manners well known in the art. The purpose for the knurled or non-smooth surfaces 22 and 24 will become clear hereinafter. As will also become apparent, conventional prior art forceps have been rongeurs cutting instruments that are not suitable for use with the present invention.

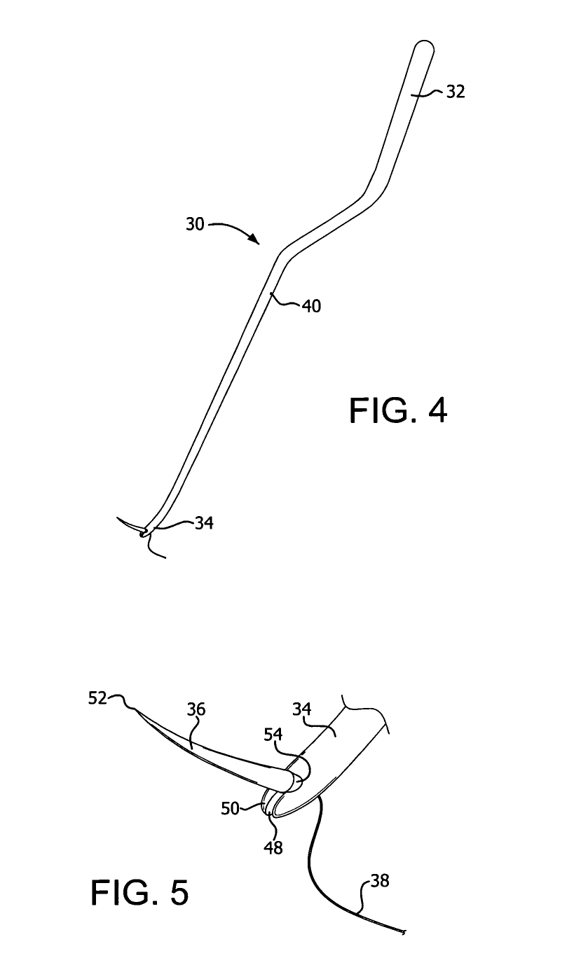

FIG. 4 illustrates the needle holder or driver instrument of the present invention which is generally designated at 30. The needle holder 30 includes a handle portion 32 which is adapted to be held by a surgeon during surgery for manipulating the instrument. The needle holder 30 also includes a needle holder portion 34 at the distal end thereof which is adapted to temporarily hold a needle 36 having a suture 38 connected thereto in a known manner.

Extending between and connecting the handle portion 32 to the needle holder portion 32 is an elongated central portion 40. The shape of the instrument 30 is commonly referred to as bayonet shaped and is particularly useful in situations where the surgical instruments cannot interfere with the surgeon's line of sight to the surgical area. In this regard, reference is made to U.S. Pat. No. 7,163,532 that illustrates one shape of an instrument that could be utilized.

As shown most clearly in FIGS. 5-9, the needle holder portion 34 of the instrument 30 includes a needle holder comprised of an aperture 42 that passes entirely through the needle holder portion 34 adjacent the distal end thereof. As best seen in FIG. 9, the aperture 42 is tapered whereby the diameter thereof is wider at the opening 44 adjacent the upper surface 46 of the needle holder portion and is narrower as the depth of the aperture increases. Thus, and as best seen in FIG. 9, the shape of the aperture 42 is essentially a truncated cone that is wider at the top than at the bottom.

The very end of the needle holder portion 34 includes a slot 48 therein that extends from the outside edge 50 of the needle holder portion 34 to the aperture 42. As will be seen, this allows the suture 38 connected to the needle 36 to pass through and into the aperture 44 without having to thread the same therethrough.

The needle 36 is preferably slightly curved as shown in FIGS. 5 and 6 and includes a pointed tip 52 at the end thereof. The needle 36 can be somewhat conventional except for the base 54 located at the other end. The base 54 is also substantially in the form of a truncated cone and is complementary to the shape of the aperture 42. As a result, the base 54 can be inserted into the aperture 42 and temporarily held therein. Because both the base 54 and the aperture 42 are tapered, the needle 36 will not pass entirely therethrough.

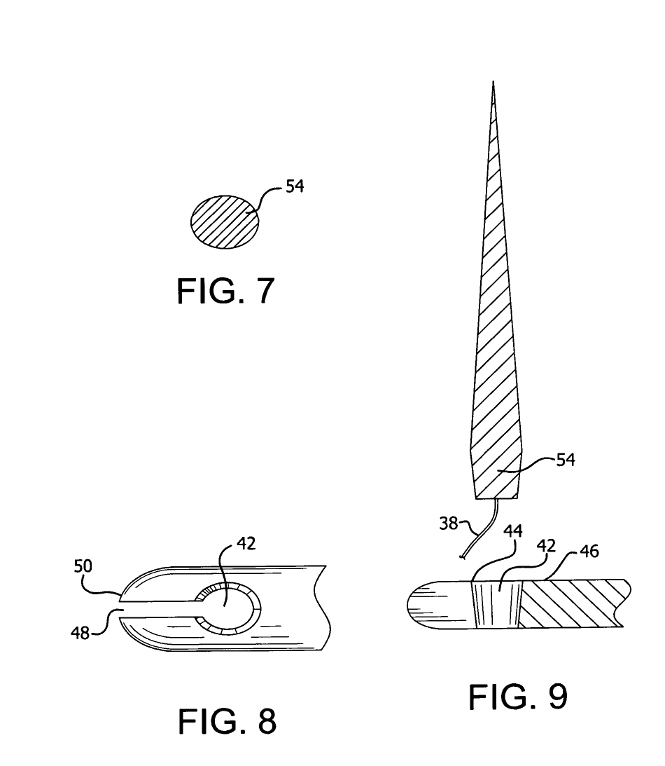

Although the aperture 42 is substantially round, it is preferably not a circle. As best shown in FIG. 8, the cross section of the opening 42 is preferably slightly ovoid in shape. And as shown in FIG. 7, the base 54 of the needle 36 is also of substantially the same ovoid shape.

The non-circular shape of the base 54 and the aperture 42 prevents the needle 36 from rotating around its own axis when it is in position in the needle holder 34. This is particularly important with curved needles so that they remain curved upwardly relative to the long axis of the instrument 30 such as shown in FIGS. 4 and 5. Furthermore, because of the shape of the base 54 and the aperture 42, the needle will be guided into its proper position as the base 54 enters the aperture 42.

The foregoing is, of course, by way of example only. There may be situations where it may be desirable to have the aperture 42 elongated in a different direction so that the needle 36 is curved or angled in a different direction. Thus, different needle or driver instruments could be provided with different needle holder apertures. Furthermore, while the aperture 42 and base 54 of the needle 36 are shown as being oval, other non-circular shapes may also be possible.

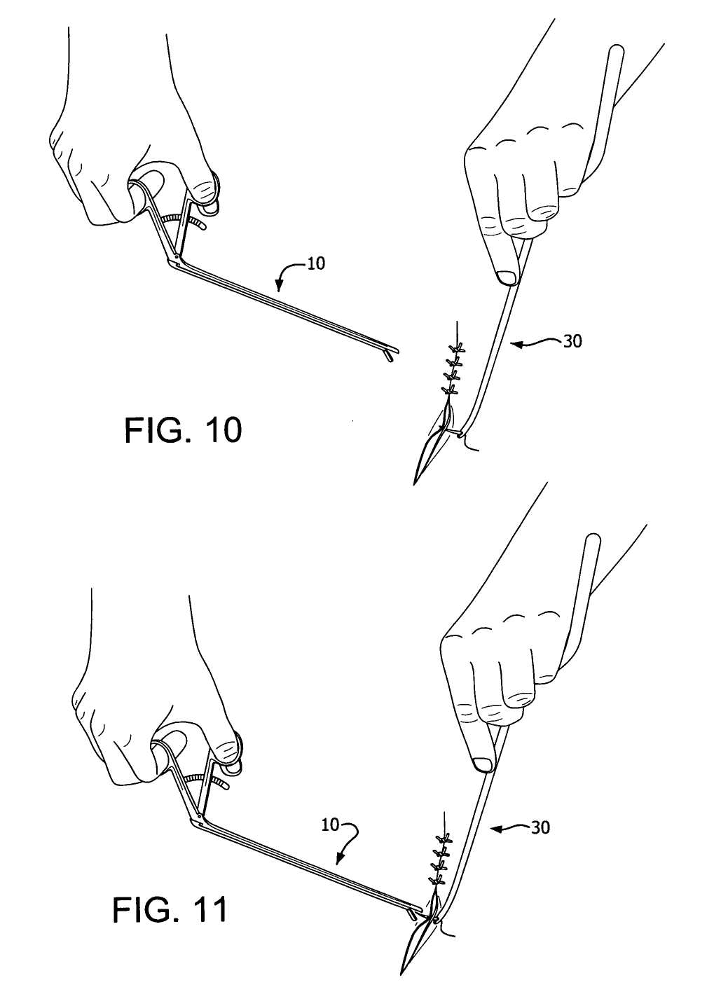

FIGS. 10, 11 and 12 illustrate the manipulative steps in the use of the instruments 10 and 30 of the present invention. Initially, a needle 36 in inserted into the aperture 42 of the needle holder or driver instrument 30 with the suture 38 extending from the back side of the needle holder. This can be done utilizing two hands or an instrument to hold the needle and place it into position. Alternatively, if the needle is held in a stationary position, the instrument 30 can be manipulated in such a way that the suture 38 passes through the slot 48 and the aperture 42 is placed over the base 54 of the needle 36 so as to position the same in place.

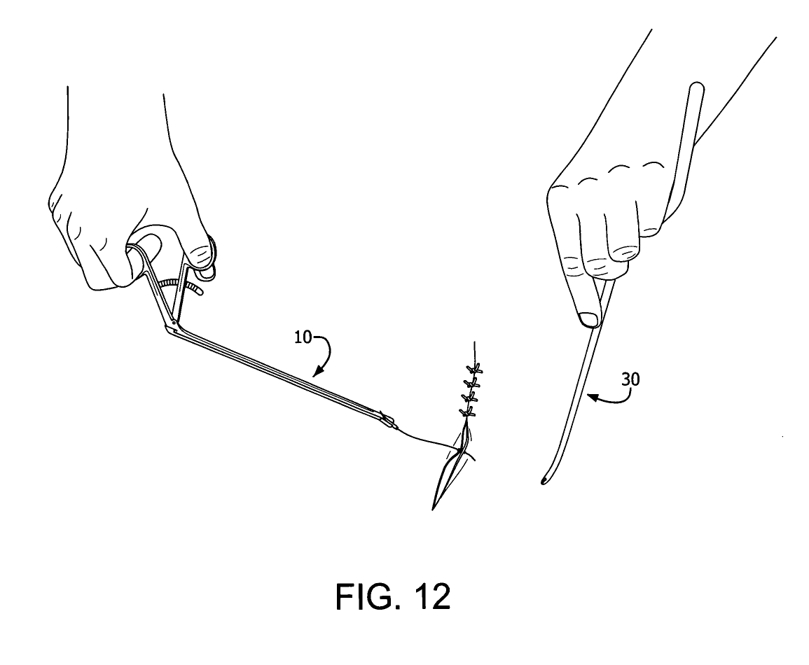

Once the needle is in position, utilizing the needle holder or driver 30, the needle is passed through the dura as shown in FIG. 10. Once it passes through the dura, the end of the needle can be grasped utilizing the jaws 18 and 20 of the forceps 10. Once the tip of the needle 36 is grasped, it is pulled through the dura as shown in FIG. 12. The process can then be repeated by again having the needle holder portion 46 grasp the base of the needle as explained above.

The present invention may be embodied in other specific forms without departing from the spirit or essential attributes thereof and accordingly, reference should be made to the appended claims rather than to the foregoing specification as indicating the scope of the invention.

We claim:

1. A needle holder instrument and needle for use in minimally invasive dural repair procedures comprising:
said needle holder instrument including a handle portion adapted to be held by a surgeon for manipulating the instrument, a needle holder portion adapted to temporarily hold a needle and an elongated central portion extending between and connecting said handle portion to said needle holder portion;
said needle holder portion including a needle holder comprised of an aperture passing entirely through said needle holder portion adjacent the distal end thereof, said aperture being tapered whereby the diameter thereof is wider at the opening adjacent the surface of said needle holder portion and is narrower as the depth of the aperture increases, and
a needle have a pointed tip at one end thereof and a base at the other end thereof, said base having a shape that is complementary to the shape of said aperture of said needle holder whereby said base can be inserted into said aperture and temporarily held therein.

2. The needle holder instrument and needle for use in minimally invasive dural repair procedures as claimed in claim 1 wherein said needle includes suture material connected thereto.

3. The needle holder instrument and needle for use in minimally invasive dural repair procedures as claimed in claim 1 wherein said needle holder portion includes a slot therein that extends from the outside edge of the needle holder portion to said aperture.

4. The needle holder instrument and needle for use in minimally invasive dural repair procedures as claimed in claim 1 wherein said aperture and said base are noncircular in cross section thereby preventing rotation of said needle relative to said needle holder.

5. A kit for use in minimally invasive dural repair procedures comprising:
a needle holder instrument, a needle and forceps;
said needle holder instrument including a handle portion adapted to be held by a surgeon for manipulating the instrument, a needle holder portion adapted to temporarily hold a needle and an elongated central portion extending between and connecting said handle portion to said needle holder portion;
said needle holder portion including a needle holder comprised of an aperture passing entirely through said needle holder portion adjacent the distal end thereof, said aperture being tapered whereby the diameter thereof is wider at the opening adjacent the surface of said needle holder portion and is narrower as the depth of the aperture increases;
said needle having a pointed tip at one end thereof and a base at the other end thereof, said base having a shape that is complementary to the shape of said aperture of said needle holder whereby said base can be inserted into said aperture and temporarily held therein, and
forceps, said forceps including opposed working jaws having non-smooth surfaces that are adapted to grasp and hold said needle.

6. A method for suturing the dura during a minimally invasive dural repair procedure including the steps of:
providing a needle holder instrument, a needle and forceps;
said needle holder instrument including a handle portion adapted to be held by a surgeon for manipulating the instrument, a needle holder portion adapted to temporarily hold said needle and an elongated central portion extending between and connecting said handle portion to said needle holder portion;
said needle holder portion including a needle holder comprised of an aperture passing entirely through said needle holder portion adjacent the distal end thereof, said aperture being tapered whereby the diameter thereof is wider at the opening adjacent the surface of said needle holder portion and is narrower as the depth of the aperture increases;
said needle having a pointed tip at one end thereof and a base at the other end thereof with suture material connected thereto, said base having a shape that is complementary to the shape of said aperture of said needle holder whereby said base can be inserted into said aperture and temporarily held therein;
said forceps including opposed working jaws having non-smooth surfaces that are adapted to grasp and hold said needle;
placing the base of said needle into said needle holder with said suture material passing through said aperture;
utilizing said needle holder, passing the tip of said needle through said dura;
utilizing said forceps, grasping the tip of said needle that has passed through said dura, drawing said needle entirely through said dura with said forceps;
replacing the base of said needle into said needle holder with said suture material passing through said aperture and again passing the tip of said needle through said dura.
